(19) United States
(12) Patent Application Publication
(10) Pub. No.: US 2007/0066842 A1
(43) Pub. Date: Mar. 22, 2007

(54) Polymerization Inhibitor Compositions, Their Preparation, and Their Use

(75) Inventor: Wenfeng Kuang, Baton Rouge, LA (US)

Correspondence Address: Albemarle Corporation, 451 Florida Street, Baton Rouge, LA, 70801-1765, US

(73) Assignee: ALBEMARLE CORPORATION, Baton Rouge, LA (US)

(21) Appl. No.: 11/533,048

(22) Filed: Sep. 19, 2006

Related U.S. Application Data
(60) Provisional application No. 60/719,053, filed on Sep. 19, 2005.

(51) Int. Cl. C07C 69/02

(52) U.S. Cl. 560/4

(57) ABSTRACT

This invention provides a polymerization inhibitor composition in the form of a solution. The solution is formed from ingredients comprised of: a) tris(N-nitroso-N-phenylhydroxylamine)aluminum salt and b) at least one liquid ethoxylated phenol acrylate of the formula:
PhO(CH₂CH₂O)ₙC(O)CH═CH₂ wherein n is an integer of 2 or more; or c) at least one liquid alkyl methacrylate or at least one liquid alkanediol dimethacrylate, or both; or d) both of b) and c); the amount of a) used in forming said solution being at least about 1 wt % based on the total weight of said solution.

### REFERENCE TO RELATED APPLICATION

This application claims the benefit and priority of U.S. Provisional Application No. 60/719,053, filed Sep. 19, 2005, the disclosure of which is incorporated herein by reference.

### TECHNICAL FIELD

This invention relates to novel stable liquid polymerization inhibitor compositions comprised of tris(N-nitroso-N-phenylhydroxylamine)aluminum salt, to methods of forming such compositions, and to use of such compositions in photocurable formulations.

### BACKGROUND

Tris(N-nitroso-N-phenylhydroxylamine)aluminum salt, CAS No. 15305-07-4, is a very effective polymerization inhibitor for use in photocurable formulations (a.k.a. radiation curable compositions.) The compound is available from Albemarle Corporation as FIRSTCURE® NPAL polymerization inhibitor. Tris(N-nitroso-N-phenylhydroxylamine)-aluminum salt is itself in the solid state of aggregation. To enhance its utility as a polymerization inhibitor, it is desirable to provide the compound in a liquid form.

Heretofore, tris(N-nitroso-N-phenylhydroxylamine)aluminum salt has been sold in a liquid polymerization inhibitor blend with the acrylic acid ester of 2-phenoxyethanol. Unfortunately however the blend has a strong unpleasant odor. Thus a need exists for a way of providing stable solutions of tris(N-nitroso-N-phenylhydroxylamine)aluminum salt that are suitable for use in forming photocurable formulations without incurring strong undesirable odor. It would be especially desirable if whatever is used to achieve these properties could also provide a stable blend in the form of a clear solution that has good storage stability, that is highly efficient in inhibiting premature polymerization in photocurable formulations in which the blend is used, and that will not adversely affect the properties of cured products formed on curing the photocurable formulation.

### BRIEF SUMMARY OF THE INVENTION

This invention provides, among other things, polymerization inhibitor compositions which possess most if not all of the foregoing attributes. In other words, the compositions are highly suitable for use as additives in forming photocurable formulations. These polymerization inhibitor compositions are in the form of a solution formed from ingredients comprised of:
a) tris(N-nitroso-N-phenylhydroxylamine)aluminum salt and
b) at least one liquid ethoxylated phenol acrylate of the formula:
   PhO(CH₂CH₂O)ₙC(O)CH═CH₂
wherein n is an integer of 2 or more; or
c) at least one liquid alkyl methacrylate or at least one liquid alkanediol dimethacrylate, or both; or
d) both of b) and c);
   the amount of a) used in forming such solution being at least about 1 wt % based on the total weight of the solution.

When c) is a combination of at least one liquid alkyl methacrylate and at least one liquid alkanediol dimethacrylate, these components can be in any proportions relative to each other, i.e., in the range of a trace of alkyl methacrylate in the combination of alkyl methacrylate and alkanediol dimethacrylate to a trace of alkanediol dimethacrylate in the combination of alkyl methacrylate and alkanediol dimethacrylate, provided the combination is liquid at least at ambient room temperatures, and preferably is liquid at temperatures below 0° C. Similarly, in the case d), components b) and c) can be in any proportions relative to each other, i.e., in the range of a trace of b) in the mixture of b) and c) to a trace of c) in the mixture of b) and c), provided the mixture of d) is liquid at least at ambient room temperatures, and preferably is liquid at temperatures below 0° C.

This invention also provides a method of producing a stable polymerization inhibitor composition in the form of a solution, which method comprises mixing together the following ingredients
a) tris(N-nitroso-N-phenylhydroxylamine)aluminum salt and
b) at least one liquid ethoxylated phenol acrylate of the formula:
   PhO(CH₂CH₂O)ₙC(O)CH═CH₂
wherein n is an integer of 2 or more; or
c) at least one liquid alkyl methacrylate or at least one liquid alkanediol dimethacrylate, or both; or
d) both of b) and c);
   to form a clear solution, the amount of a) used in forming said solution being at least about 1 wt % based on the total weight of said solution. Storing such clear solution results in a longer period during which the solution remains solids-free as compared to storing neat tris(N-nitroso-N-phenylhydroxylamine)aluminum salt under the same storage conditions.

Other embodiments and features of this invention will be further apparent from the ensuing description and appended claims.

### FURTHER DETAILED DESCRIPTION OF THE INVENTION

1) Polymerization Inhibitor Compositions

As can be seen from the above, this invention makes available a variety of polymerization inhibitor compositions. One type of novel polymerization inhibitor compositions is in the form of solutions formed from ingredients comprised of tris(N-nitroso-N-phenylhydroxylamine)aluminum salt with at least one liquid ethoxylated phenol acrylate of the formula:
PhO(CH₂CH₂O)ₙC(O)CH═CH₂
wherein n is an integer of 2 or more, the amount of tris(N-nitroso-N-phenylhydroxylamine)-aluminum salt a) used in forming such solutions being at least about 1 wt % based on the total weight of the solution. Typically n is in the range of 2 to about 20. Preferably n is in the range of 2 to about 6. An especially preferred embodiment involves blends of these components wherein n is such that there is either (i) a single compound in which n is 2 or (ii) a liquid mixture of two or more ethoxylated phenol acrylates of the above formula where n in at least 95% of the liquid mixture of ethoxylated phenol acrylates is in the range of 2 to about 6 with the average of n for the mixture being in the range of 2 to about 4. As between (i) and (ii), use of (i) is more preferred.

Another type of novel polymerization inhibitor compositions made available by this invention is in the form of solutions formed from ingredients comprised of tris(N-nitroso-N-phenylhydroxylamine)aluminum salt with at least one liquid alkyl methacrylate, preferably methyl methacrylate, the amount of tris(N-nitroso-N-phenylhydroxylamine)aluminum salt used in forming such solutions being at least about 1 wt % based on the total weight of the solution. In lieu of or in addition to use of at least one liquid alkyl methacrylate, at least one liquid alkanediol dimethacrylate can be employed in accordance with this type of polymerization inhibitor composition.

A third type of novel polymerization inhibitor compositions of this invention is in the form of solutions formed from ingredients comprised of tris(N-nitroso-N-phenyl-hydroxylamine)aluminum salt with (A) at least one ethoxylated phenol acrylate of the above first embodiment and (B) at least one liquid alkyl methacrylate or at least one liquid alkanediol dimethacrylate, or both, of the above second embodiment, wherein the combination of (A) and (B) is a liquid, the amount of tris(N-nitroso-N-phenylhydroxylamine-)aluminum salt used in forming such solutions being at least about 1 wt % based on the total weight of the solution.

The polymerization inhibitor compositions of this invention have good solubilities in common organic solvents and media, and good storage stabilities, and are highly efficient in inhibiting premature polymerization of various photocurable formulations in which they can be used. Moreover the components of these blends can be proportioned so as to provide little if any offensive odor.

The polymerization inhibitor compositions of this invention contain at least about 1 wt %, typically at least about 3 wt %, preferably at least about 5 wt %, and more preferably at least about 6 wt % of dissolved tris(N-nitroso-N-phenylhydroxylamine)aluminum salt based on the total weight of the composition. The components of the blends are proportioned such that they do not exceed the room temperature (e.g., 23° C.) solubility limit of tris(N-nitroso-N-phenylhydroxylamine)aluminum salt in the particular solvent(s) used in forming the blend. However in instances where product appearance is not a material factor, the blends may even be concentrated cloudy liquids.

Preparation of tris(N-nitroso-N-phenylhydroxylamine)aluminum salt is known in the art and is described for example in U.S. Pat. No. 6,018,078. For example, tris(N-nitroso-N-phenylhydroxylamine)aluminum salt can be prepared by reacting an aluminum salt, such as aluminum sulfate, with N-nitroso-N-phenylhydroxylamine ammonium salt, although other salts, such as halides, phosphates, nitrates, carbonates, and organic acid salts of the metal can also be used.

To form the polymerization inhibitor compositions of this invention, the components thereof are mixed together in any suitable manner using typical mixing apparatus such as a blending tank or vessel equipped with suitable agitation or stirring means.

Additional components may be included in the polymerization inhibitor compositions of this invention, provided that such components do not materially detract from the beneficial attributes of compositions in which they are employed. Non-limiting examples of additional components which may be included in the blends of this invention include dyes, acrylate and/or methacrylate monomers other than those specified above, acrylate and/or methacrylate oligomers, common organic solvents, oxidation inhibitors, viscosity modifiers, fillers, photoinitiators, and tertiary amines. Specific non-limiting examples of such additional components include acetone, isopropyl alcohol, acetonitrile, waxes, polymer powders, N,N-diethyl-4-aminoazobenzene (DEAB), (2,4,6 trimethyl benzoyl)diphenyl phosphine oxide (TPO, Albemarle Corporation), 2-methyl-[4-(methylthio)phenyl]-2-morpholinopropan-1-one (Irgacure® 907, Ciba Chemical Specialties), phenylbis(2,4,6-trimethylbenzoyl)-phosphine oxide (Irgacure® 819, Ciba Chemical Specialties), liquid aliphatic amine synergist (FIRSTCURE® AS-5 amine, Albemarle Corporation), ethyl-4(dimethylamino)benzoate (EDAB), 2-ethylhexyl-4(dimethylamino)benzoate (ODAB, Albemarle Corporation), N-methyl-N,N-diethanolamine, tris-(2,4-di-t-butylphenyl)phosphite (ETHAPHOS™ 368, Albemarle Corporation), and octadecyl-3-(3,5-di-t-butyl-4-hydroxyphenyl)-propionate (ETHANOX® 376, Albemarle Corporation).

When additional components are used in forming polymerization inhibitor compositions of this invention, the resultant liquid polymerization inhibitor composition should contain at least 10 wt % of the combination of a) tris(N-nitroso-N-phenylhydroxylamine)aluminum salt with b) at least one liquid ethoxylated phenol acrylate of the formula:
PhO(CH₂CH₂O)ₙC(O)CH═CH₂
wherein n is an integer of 2 or more; or c) at least one liquid alkyl methacrylate; or at least one liquid alkanediol dimethacrylate, or both, or d) both of b) and c).

Preferred polymerization inhibitor compositions of this invention are formed from components which—except for traces of impurities that are or may be normally present in the components themselves or traces of impurities resulting from contact with containers, stirring blades and/or shafts, conduits or pipes, or similar equipment encountered in connection with preparation, transportation, and/or storage of the components or the polymerization inhibitor compositions themselves—are composed entirely or substantially entirely (e.g., at least 10 wt % and preferably at least 20 wt % of a) tris(N-nitroso-N-phenylhydroxylamine)aluminum salt and b) at least one liquid ethoxylated phenol acrylate of the formula:
PhO(CH₂CH₂O)ₙC(O)CH═CH₂
wherein n is an integer of 2 or more; or c) at least one liquid alkyl methacrylate or at least one liquid alkanediol dimethacrylate, or both, or d) both of b) and c).

Any liquid alkyl methacrylate or liquid mixture of alkyl methacrylates can be used as component c) in forming the polymerization inhibitor compositions of this invention. The alkyl group of such liquid alkyl methacrylates typically contains in the range of 1 to about 12 carbon atoms. Preferred liquid alkyl methacrylates contain in the range of 1 to about 6 carbon atoms in the alkyl group. Methyl methacrylate is particularly preferred.

Similarly, any liquid alkanediol dimethacrylate or liquid mixture of alkanediol dimethacrylates can be used as component c) in forming the polymerization inhibitor compositions of this invention. Such liquid alkanediol dimethacrylates typically contain in the range of 2 to about 8 carbon atoms in the alkylene group. Preferred liquid alkanediol dimethacrylates contain in the range of 2 to about 6 carbon atoms in the alkylene group. Ethylene glycol dimethacrylate, 1,4-butanediol dimethacrylate, 1,6-hexanediol dimethacrylate,1,8-octanediol dimethacrylate, and glycerol dimethacrylate serve as non-limiting examples of the alkanediol dimethacrylates that may be used as component c) in the practice of this invention.

2) Photocurable Formulations

The polymerization inhibitor compositions of this invention can be used as polymerization inhibitors in, or in forming, any of a wide variety of photocurable formulations. For example, a photocurable formulation formed using a polymerization inhibitor composition can contain in addition, one or more photocurable monomers, one or more photocurable oligomers or polymers, or one or more photoinitiators (free-radical or cationic). Among additional components, one or more of which can also be in such formulations are, for example, coinitiators (a.k.a. synergists), rheology modifiers, pigments, dyes, light stabilizers, radical scavengers and adhesion promoters. The composition and use of such substances are well known to those of skill in the art, are described in the literature, and numerous products serving these functions in photocurable formulations are available in the marketplace.

Photocurable formulations produced by use of polymerization inhibitor compositions of this invention can be formed by mixing a blend of this invention with the ingredients typically used in forming a radiation curable formulation. Mixing apparatus such as a blending tank or vessel equipped with suitable agitation or stirring means can be used in forming the radiation curable formulations of this invention. Photocurable formulations produced by use of polymerization inhibitor compositions of this invention typically contain in the range of about 0.005 to about 10 wt %, and preferably in the range of about 0.1 to about 4 wt %, of a blend of this invention, based on the total weight of the formulation.

A few non-limiting examples of photocurable monomers, oligomers, and/or polymers that can be used in forming photocurable formulations include monomeric, oligomeric, polymeric, or copolymeric forms of methyl acrylate, methyl methacrylate, ethyl acrylate, ethyl methacrylate, butyl acrylate, butyl methacrylate, 2-ethylhexyl acrylate, 2-ethylhexyl methacrylate, lauryl acrylate, lauryl methacrylate, cyclohexyl acrylate, cyclohexyl methacrylate, isobornyl acrylate, isobornyl methacrylate, hydroxyethyl acrylate, hydroxyethyl methacrylate, dimethylaminopropyl acrylate, dimethylaminopropyl methacrylate, diethylaminopropyl acrylate, diethylaminopropyl methacrylate, and the like, as well as mixtures of any two or more thereof.

Preferred photocurable monomers, oligomers, and/or polymers which may be used in such formulations include monomeric, oligomeric, polymeric, or copolymeric forms of tripropylene glycol diacrylate, trimethylol propane tetraacrylate, ethoxylated trimethylol propane tetraacrylate, prop oxylated neopentyl glycol diacrylate, hex anediol diacrylate, and the like, as well as mixtures of any two or more thereof.

If desired, alpha,beta-ethylenically unsaturated carboxylic acids can be used in conjunction with acrylate and/or methacrylate monomers, oligomers, and/or polymers, typically for the purpose of providing improved adhesion to certain substrates. Examples of such acids include methacrylic acid, acrylic acid, itaconic acid, maleic acid, beta-carboxyethyl acrylate, beta-carboxyethyl methacrylate, and the like, as well as mixtures of any two or more thereof.

Other photocurable monomers, oligomers, and/or polymers which may be used in such formulations include monomeric, oligomeric, polymeric, or copolymeric forms of thiols, vinyl ethers, vinyl esters, N-vinyl compounds, unsaturated polyesters, styrene, divinyl benzene, acrylonitrile, polyester acrylates, polyurethane acrylates, epoxy acrylates, polyether acrylates, silicone acrylates, fluoro-acrylates, and amine acrylates.

Formulations to be subjected to photocuring typically contain in the range of about 0.5 to about 85 wt % of one or more photocurable monomers, oligomers, or polymers such as those described above. Preferred formulations contain in the range of about 20 to about 75 wt % of one or more of such photocurable substances. Selections within these ranges are typically made for effecting adjustments of viscosity to suit the particular application method to be used. For example, photocurable formulations adapted for use in forming low viscosity web coatings, typically contain in the range of about 50 to about 70 wt % of one or more such monomers, oligomers, or polymers based on the weight of the total composition to be subjected to photocuring (radiation curing).

Free-radical photoinitiators which can be used in the photocurable formulations that can be formed using the polymerization inhibitor compositions of this invention are of two general types: Type I, those that undergo photocleavage to yield free-radicals and Type II, those that produce initiating radicals through an abstraction process. Type I photoinitiators produce radicals through a unimolecular fragmentation. Examples of these include aromatic carbonyl compounds, such as derivatives of benzoin, benzilketal and acetophenone. Type II (abstraction type) photoinitiators are typically aromatic ketones, such as thioxanthones and benzophenone derivatives. In these systems, a coinitiator must be present in order to produce an initiating radical. These coinitiators can include amines, alcohols, thiols, or ethers. The process of producing radicals is either through a hydrogen abstraction or an electron transfer mechanism depending on the coinitiator.

Non-limiting examples of photoinitiators that can be used in the radiation curable formulations include Type I (unimolecular fragmentation type) initiators, such as alpha-diketone compounds or monoketal derivatives thereof (e.g., diacetyl, benzil, benzyl, or dimethylketal derivatives); acyloins (e.g., benzoin, pivaloin, etc.); acyloin ethers (e.g., benzoin methyl ether, benzoin ethyl ether, benzoin propyl ether, etc.), acyl phosphine oxides, and other similar Type I initiators, including mixtures of any two or more such initiators. Similarly, Type II (abstraction-type) initiators can be used. Non-limiting examples of suitable Type II initiators include xanthone, thioxanthone, 2-chloroxanthone, benzil, benzophenone, 4,4′-bis(N,N′-dimethylamino)benzophenone, polynuclear quinones (e.g., 9,10-anthraquinone, 9,10-phenanthrenequinone, 2-ethyl anthraquinone, and 1,4-naphthoquinone), or the like, as well as mixtures of any two or more thereof. Preferred Type I initiators include ketals such as benzyl dimethyl ketal. Preferred Type II initiators include hydrogen quinones such as benzoquinone and 2-ethyl anthraquinone. Mixtures of Type I and Type II initiators can also be used. The initiator or mixture of initiators is typically added in an amount of about 0.01 to about 10 parts by weight, preferably about 0.05 to about 5 parts by weight, per 100 parts by weight of the monomer(s), oligomer(s), or polymer(s) to be photocured.

Of the various coinitiators that can be used in the formulations, tertiary amines are typically favored for use. A few non-limiting examples of amine coinitiators which can be used in the formulations include N [3-(dimethylamino)propyl]-N,N′,N′-trimethyl-1,3-propanediamine (Polycat 77; Air Products, Inc.), 2,2′-oxybis[N,N-dimethylethanamine] (DABCO BL-19; Air Products, Inc.), N,N-dimethyl-4-morpholineethanamine (DABCO XDM; Air Products, Inc.); methyldiethanolamine; acrylate amines; 2-ethylhexyl-4(dimethylamino)benzoate (ODAB); ethyl-4(dimethylamino)benzoate (EDAB); morpholine derivatives; and one or more trialkylamines each having a total of 10 to about 36 carbon atoms in the molecule and wherein at least one alkyl group has a chain length of at least 8 carbon atoms, e.g., (A) one or more trialkyl amines each having a total of 10 to about 24 carbon atoms in the molecule, and wherein two of the alkyl groups are methyl or ethyl, or one of each, (more preferably both are methyl), and the remaining alkyl group contains at least 8 carbon atoms (and more preferably is a primary alkyl group containing in the range of 8 to about 22 carbon atoms), or (B) one or more trialkyl amines each having a total of 17 to about 38 carbon atoms in the molecule, and wherein one of the alkyl groups is methyl or ethyl (preferably methyl), and the other two alkyl groups are the same or different, and each is a primary alkyl group). Dodecyldimethylamine, tetradecyldimethylamine, hexadecyldimethyl-amine, octadecyldimethylamine, didecylmethylamine, and dodecylmethylamine are illustrative of tertiary amines of (A) or (B). Mixtures of two or more tertiary amine coinitiators can be used. Amounts of amines typically are in the range of about 0.5 to about 40 wt % of the total weight of the formulation.

Non-limiting examples of rheology modifiers which can be used in forming the formulations include such materials as organic silicone compounds, organic fluoro-compounds, polyethers, and organic ionic compounds. Typically, rheology modifiers are used in amounts in the range of about 0.01 to about 5 wt % of the formulation.

Pigments and dyes can be used, and often are preferably used, in photocurable formulations. Examples of pigments and typical amounts used in the formulation include phthalocyanine blue (about 5 to about 20 wt %), titanium dioxide (about 10 to about 30 wt %), or other organic or inorganic pigments employed in the art. Optionally, dyes such as nigrosine black or methylene blue may be used to enhance color or tone (e.g., about 1 to about 5 wt %).

Light stabilizers are another type of additives which can be used in the photocurable formulation in which a polymerization inhibitor composition of this invention is employed. Non-limiting examples of such light stabilizers include 2-hydroxybenzophenones such as 2,2′-dihydroxy-4,4′-dimethoxylbenzophenone, 2-(2-hydroxyphenyl)benzotriazoles such as 2-(2′-hydroxyphenyl)benzotriazole, sterically-hindered amines such as bis(2,2,6,6-tetramethyl-4-piperidyl)sebacate or bis(2,2,6,6-tetramethyl-4-piperidyl)succinate, oxamides such as 4,4′-dioctyloxyanilide, acrylates such as ethyl α-cyano-β,β-diphenylacrylate or methyl α-carbomethoxycinnanamate, and nickel complexes such as the nickel complex of 2,2′-thiobis[(1,1,3,3-tetramethylbutyl)phenol. Typically the amount used will be in the range of about 0.02 to about 5 wt % depending upon the particular type of light stabilizer employed.

Still another type of additives which can be used in forming radiation curable compositions is one or more radical scavengers. Non-limiting examples of suitable radical scavengers for such use include hydroquinone, hydroquinone methyl ether, p-tert-butylcatechol, quinoid compounds such as benzoquinone and alkyl-substituted benzoquinones, as well as other radical scavenger compounds known in the art. Typically these components will be used in amounts in the range of about 100 ppm to about 2 percent by weight of the composition.

Adhesion promoters constitute yet another type of additive components which can be used in the formation of photocurable compositions in which a polymerization inhibitor composition of this invention is employed. Such components typically are silane derivatives such as gamma-aminopropyltriethoxysilane (DOW A-1100) and equivalent substituted silane products; acid functionally-substituted resins; oligomers or monomers, such as partial esters of phosphoric acid, maleic anhydride, or phthalic anhydride, with or without acrylic or methacrylic unsaturation; and dimers and trimers of acrylic/methacrylic acid. If adhesion promoters are used, the preferred types are other than alpha,beta-ethylenically unsaturated carboxylic acids. If and when used, the concentration thereof is determined empirically by adhesion tests. In general, however, amounts are often in the range of about 0.5 to about 20 wt %, and in more preferred cases in the range of about 2 to about 10 wt % of the total weight of the composition.

In effecting photocuring either coherent or non-coherent radiation can be employed. Various sources of such radiation can be employed, such as an ion gas laser (e.g., an argon ion laser, a krypton laser, a helium:cadmium laser, or the like), a solid state laser (e.g., a frequency-doubled Nd:YAG laser), a semiconductor diode laser, an arc lamp (e.g., a medium pressure mercury lamp, a Xenon lamp, or a carbon arc lamp), and like radiation sources. Exposure sources capable of providing ultraviolet and visible wavelength radiation (with wavelengths typically falling in the range of 200-700 nm) can also be used for conducting the photocuring. Preferred wavelengths are those which correspond to the spectral sensitivity of the initiator being employed. Preferred radiation sources are gas discharge lamps using vapors of mercury, argon, gallium, or iron salts and utilizing magnetic, microwave or electronic ballast; such lamps commonly are medium pressure mercury lamps, or lamps made by Fusion Systems (i.e., D, H, and V lamps).

Exposure times can vary depending upon the radiation source, and photoinitiator(s) being used. For high speed applications such as in forming thin coatings on paper webs traveling at high linear speeds, times in the range of about 0.005 to about 0.015 second are desirable. In radiation curing (photopolymerization) operations in which the mixture being polymerized is either stationary or moving slowly as on a conveyor belt, longer exposure times (e.g., in the range of about 0.2 to about 0.4 second can be used.

There are various known ways of conducting photocuring operations. For example, a photocurable formulation can be photopolymerized as a thin coating on a traveling web. Alternatively, the radiation curable formulation can be photopolymerized or photocured as a coating or laminate on a substrate. Another variant is where the radiation curable formulation is photopolymerized as an article or shape while in a mold. In these and other modes of operation, the exposure to radiation for effecting curing or polymerization can be continuous or intermittent.

Various radiation cured articles and shapes can be produced by radiation curing of a photopolymerizable composition formed using a polymerization inhibitor of this invention. Thus the radiation cured (photopolymerized) end product can be printed matter on a substrate such as paper, cardboard, or plastic film, etc.; manufactured articles such as handles, knobs, inkstand bases, small trays, rulers, etc.; and coatings or laminates on substrates such as plywood, metal sheeting, polymer composite sheeting, etc. As noted above, thin coated paper and coated card or thin paperboard stock where the coatings are in the range of about 0.02 to about 10 mils in thickness constitute desirable articles produced from the photocurable compositions.

The following examples illustrate the practice and some of the advantages of this invention. The Examples are not intended to limit, and should not be construed as limiting, the generic scope of this invention.

Typical properties or characteristics as provided by the manufacturers of materials used in the following Examples are as follows:

FIRSTCURE® NPAL Polymerization Inhibitor; Albemarle Corporation—

tris(N-nitroso-N-phenylhydroxylamine)aluminum salt having the empirical formula C₁₈H₁₅N₆O₆Al. It is an off-white powder with a molecular weight of 468 and a melting point range of 165-170° C.
   Ebecryl® 110; Cytec Surface Specialities—
acrylic acid ester of 2-phenoxyethoxyethanol having a molecular weight of 236, a viscosity at 25° C. of 22 cP, and a density of about 1.12.
   OTA-480; Cytec Surface Specialities—
propoxylated glycerol triacetate monomer having a molecular weight of 480, a viscosity at 25° C. of 85 cP, and a density of about 1.08.
   Ebecryl® 3720; Cytec Surface Specialities—
bisphenol-A epoxy diacrylate having a molecular weight of 500, a viscosity at 65.5° C. of 2000 cP.

### EXAMPLE 1

A polymerization inhibitor composition of this invention was formed by preparing a solution containing 6 wt % of tris(N-nitroso-N-phenylhydroxylamine)aluminum salt (FIRSTCURE® NPAL Polymerization Inhibitor; Albemarle Corporation) and 94 wt % of an acrylic acid ester of 2-phenoxyethoxyethanol (Ebecryl® 110; Cytec Surface Specialities). The resultant liquid composition was stable at temperatures as low as 12° F. (ca. 10.1° C.) for two days (no crystals were visually perceptible), and was free of disagreeable odor.

### COMPARATIVE EXAMPLE A

A blend not of this invention was formed by preparing a mixture containing 6 wt % of tris(N-nitroso-N-phenylhydroxylamine)aluminum salt (FIRSTCURE® NPAL Polymerization Inhibitor; Albemarle Corporation) and 94 wt % of a propoxylated glycerol triacetate monomer (OTA-480; Cytec Surface Specialities). The resultant liquid blend contained a crystalline precipitate which remained insoluble in the blend at room temperature.

### EXAMPLE 2

A polymerization inhibitor composition of this invention was formed from the ingredients given in Table 1.

TABLE 1

| Components | Weight |
|---|---|
| Tris(nitroso-N-phenylhydroxylamine)aluminum salt | 6 |
| Acrylic acid ester of 2-phenoxyethoxyethanol (Ebecryl ® 110) | 30 |
| Bisphenol-A epoxy diacrylate (Ebecryl ® 3720) | 32 |
| Propyloxylated glycerol triacrylate (OTA-480) | 20 |
| Hydroquinone monomethyl ether (MEHQ) | 3 |
| 2,6-Di-tert-butyl-p-cresol | 9 |

The blend was a clear solution containing 6 wt % of tris(nitroso-N-phenylhydroxylamine)aluminum salt, and with the acrylic acid ester of 2-phenoxyethoxyethanol and tris(nitroso-N-phenylhydroxylamine)aluminum salt used in a weight ratio of 5:1, respectively.

### COMPARATIVE EXAMPLE B

A polymerization inhibitor composition not of this invention was formed from the ingredients given in Table 2. This blend was devoid of any liquid ethoxylated phenol acrylate.

TABLE 2

| Components | Weight |
|---|---|
| Tris(nitroso-N-phenylhydroxylamine)aluminum salt | 4 |
| Bisphenol-A epoxy diacrylate (Ebecryl ® 3720) | 32 |
| Propyloxylated glycerol triacrylate (OTA-480) | 50 |
| Hydroquinone monomethyl ether (MEHQ) | 4 |
| 2,6-Di-tert-butyl-p-cresol | 10 |

The polymerization inhibitor blend of Example 2 was found to be substantially more efficient at stabilizing a radiation curable formulation stored at ambient room temperature or at elevated temperatures (e.g., 60° C.) than the blend of the Comparative Example B.

It is to be understood that the ingredients referred to by chemical name or formula anywhere in the specification or claims hereof, whether referred to in the singular or plural, are identified as they exist prior to coming into contact with another substance referred to by chemical name or chemical type (e.g., another reactant, a solvent, a diluent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and other materials are identified as ingredients to be brought together in connection with performing a desired chemical reaction or in forming a mixture to be used in conducting a desired reaction. Also, even though the claims hereinafter may refer to substances, components and/or ingredients in the present tense (“comprises”, “is”, etc.), the reference is to the substance or ingredient as it existed at the time just before it was first contacted, blended or mixed with one or more other substances or ingredients in accordance with the present disclosure. The fact that the substance or ingredient may have lost its original identity through a chemical reaction or transformation or complex formation or assumption of some other chemical form during the course of such contacting, blending or mixing operations, is thus wholly immaterial for an accurate understanding and appreciation of this disclosure and the claims thereof. Nor does reference to an ingredient by chemical name or formula exclude the possibility that during the desired reaction itself an ingredient becomes transformed to one or more transitory intermediates that actually enter into or otherwise participate in the reaction. In short, no representation is made or is to be inferred that the named ingredients must participate in the reaction while in their original chemical composition, structure or form.

Each and every patent or other publication or published document referred to in any portion of this specification is incorporated in toto into this disclosure by reference, as if fully set forth herein.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove. Rather, what is intended to be covered is as set forth in the ensuing claims.

That which is claimed is:

1. A polymerization inhibitor composition in the form of a solution formed from ingredients comprised of:
a) tris(N-nitroso-N-phenylhydroxylamine)aluminum salt and
b) at least one liquid ethoxylated phenol acrylate of the formula:
PhO(CH₂CH₂O)ₙC(O)CH═CH₂
wherein n is an integer of 2 or more; or
c) at least one liquid alkyl methacrylate or at least one liquid alkanediol dimethacrylate, or both; or
d) both of b) and c);
the amount of a) used in forming said solution being at least about 1 wt % based on the total weight of said solution.

2. A composition as in claim 1 wherein said solution is formed from ingredients a) and b) without use of c) or d).

3. A composition as in claim 1 wherein said solution is formed from ingredients a) and b) without use of c) or d), and wherein n is 2.

4. A composition as in claim 1 wherein said solution is formed from ingredients a) and c) without use of b) or d).

5. A composition as in claim 1 wherein said solution is formed from ingredients a) and c) without use of b) or d), and wherein c) is methyl methacrylate.

6. A composition as in claim 1 wherein said solution is formed from ingredients a) and d).

7. A composition as in claim 1 wherein said solution is formed from ingredients a) and d), and wherein n is 2.

8. A composition as in claim 1 wherein said solution is formed from ingredients a) and d), and wherein c) is methyl methacrylate.

9. A composition as in claim 1 wherein said amount of a) is at least about 3 wt % based on the total weight of said solution.

10. A composition as in claim 1 wherein said amount of a) is at least about 5 wt % based on the total weight of said solution.

11. A composition as in claims 2 or 3 wherein said amount of a) is at least about 6 wt % based on the total weight of said solution.

12. A composition as in claim 2 wherein bisphenol-A epoxy diacrylate and propoxylated glycerol triacrylate monomer are also ingredients used in the formation of said solution.

13. A method of producing a stable polymerization inhibitor composition in the form of a solution, which method comprises mixing together the following ingredients
a) tris(N-nitroso-N-phenylhydroxylamine)aluminum salt and
b) at least one liquid ethoxylated phenol acrylate of the formula:
PhO(CH₂CH₂O)ₙC(O)CH═CH₂
wherein n is an integer of 2 or more; or
c) at least one liquid alkyl methacrylate or at least one liquid alkanediol dimethacrylate, or both; or
d) both of b) and c);
to form a clear solution, the amount of a) used in forming said solution being at least about 1 wt % based on the total weight of said solution.

14. A method as in claim 13 wherein said amount of a) is at least about 3 wt % based on the total weight of said solution.

15. A method as in claim 13 wherein said amount of a) is at least about 5 wt % based on the total weight of said solution.

16. A method as in claim 13 wherein ingredients a) and b) are used without use of c) or d) in forming said solution.

17. A method as in claim 13 wherein ingredients a) and b) are used without use of c) or d) in forming said solution, and wherein n is 2.

18. A method as in claim 13 wherein ingredients a) and c) are used without use of b) or d) in forming said solution.

19. A method as in claim 13 wherein ingredients a) and c) are used without use of b) or d) in forming said solution, and wherein c) is methyl methacrylate.

20. A method as in claim 13 wherein ingredients a) and d) are used in forming said solution.

21. A method as in claim 13 wherein ingredients a) and d) are used in forming said solution, and wherein n is 2.

22. A method as in claim 13 wherein ingredients a) and d) are used in forming said solution, and wherein c) is methyl methacrylate.

23. A method as in claim 16 wherein said amount of a) is at least about 6 wt % based on the total weight of said solution.

24. A method as in claim 17 wherein said amount of a) is at least about 6 wt % based on the total weight of said solution.

25. In the preparation of a photocurable formulation containing a polymerization inhibitor, the improvement wherein said polymerization inhibitor introduced into, or utilized in preparing, said formulation is a polymerization inhibitor as in any of claims 1, 2, 4, 6, or 12.

26. The improvement as in claim 25 wherein the amount of a) used in forming said solution is at least about 3 wt % based on the total weight of said solution.

27. The improvement as in claim 25 wherein the amount of a) used in forming said solution is at least about 5 wt % based on the total weight of said solution.

28. The improvement as in claim 25 wherein said solution is formed from ingredients a) and b) without use of c) or d), and wherein the amount of a) used in forming said solution is at least about 6 wt % based on the total weight of said solution.
